(19) United States
(12) Patent Application Publication
(10) Pub. No.: US 2005/0143637 A1
(43) Pub. Date: Jun. 30, 2005

(54) Disposable tip for tonometer

(75) Inventors: Steven E. Feldon, Rochester, NY (US); Bruce Allen Burr, Tustin, CA (US); Richard Wademan Teasdale, Torrey, UT (US); Gregory John Netherwood, Orange, CA (US)

Correspondence Address: ORRICK, HERRINGTON & SUTCLIFFE, LLP, 4 PARK PLAZA, SUITE 1600, IRVINE, CA, 92614-2558, US

(73) Assignee: MARCO OPHTHALMIC, INC.

(21) Appl. No.: 10/855,288

(22) Filed: May 26, 2004

Related U.S. Application Data
(60) Provisional application No. 60/532,485, filed on Dec. 24, 2003.

(51) Int. Cl. A61B 3/16

(52) U.S. Cl. 600/406; 600/405

(57) ABSTRACT

There is disclosed herein an applanation tonometer and more particularly a new form of disposable tip cover therefor. The tonometer includes a fiber optic array cylinder. A disposable tip cover comprising a translucent and light diffusing film is used on the exterior tip of the cylinder. The tip cover can be retained on the cylinder by sandwiching the cover between a pair of inner and outer flexible rings, the inner ring being attachable to the tip of the fiber optic array cylinder.

The present application is related to U.S. patent application Ser. No. 10/137,234 filed Apr. 30, 2002, the disclosure of which is fully incorporated herein by reference. The present application is a regular utility application claiming priority from U.S. provisional patent application Ser. No. 60/532,485, filed Dec. 24, 2003, the disclosure of which is fully incorporated herein by reference.

### BACKGROUND OF THE INVENTION

The invention relates to the measurement of intraocular pressure and, in particular, to methods and apparatus for measuring intraocular pressure using applanation tonometry.

The measurement of intraocular pressure (IOP) is essential to the diagnosis and management of glaucoma, a major cause of blindness in the United States and around the world. Although direct measurement of intraocular pressure can be obtained by inserting a pressure sensitive probe into the eye, clinical methods must rely on indirect methods of obtaining intraocular pressure. There are two popular methods for obtaining these indirect measurements. In a first method, the eye is indented using a tonometer, popularized by Schiotz, wherein gram weights are placed on a central post that exerts pressure on a globe relative to a curved plate that covers an anesthetized cornea. This method has several disadvantages, which include the requirement that the patient remain in a supine position in addition to errors in measurement related to scleral rigidity. Also, the Schiotz tonometer requires a normogram to interpret the measured pressure.

The second method for obtaining an indirect measurement of intraocular pressure is the applanation technique, wherein a portion of the cornea is flattened by a mechanical device. In applanation techniques, the force required to produce a flattening of the cornea is related to intraocular pressure, so the intraocular pressure can be determined indirectly by measuring the flattening of the cornea and the pressure required to produce that flattening. Goldmann determined the exact area required such that one gram of force is equivalent to one mm Hg of intraocular pressure. McKay and Marg developed an electronic tonometer based on differential applanation between a central post and a surrounding annulus. This principle is utilized by the TONO-PEN™ electronic tonometer, patented by Feldon et al. in U.S. Pat. No. 4,747,296. A variant of the applanation methodology requires no direct contact with the patient's eye. This “air puff” technique involves directing a calibrated packet of pressurized air onto the corneal surface, which causes corneal flattening. This corneal flattening is then measured indirectly by measuring the deflection of light reflected from the corneal surface.

Of the various types of tonometers available for clinical use, the Goldmann applanation tonometer is considered the “gold standard.” However, there are several shortcomings to this technique. First, the device, as originally designed, is not portable, but is attached to a slit lamp. This issue has been addressed by the Perkins and Kowa tonometers that incorporate a Goldmann-type tonometer in a portable design. Second, these tonometers are manual devices that rely upon a highly trained observer to obtain reliable results. Third, the prolonged amount of time required to position the patient as well as poor patient tolerance make Goldmann tonometry inefficient and sometimes impossible to perform. Finally, a Goldmann tonometer touches the eye with a non-disposable device that is difficult to sterilize between uses. This increases the likelihood of transmitting infectious diseases or causing chemical damage to the cornea from residual antiseptic coming into contact with the patient's eye.

### SUMMARY OF THF INVENTION

An applanation tonometer and method for measuring intraocular pressure are described herein, and particularly a new disposable tip for the tonometer. The invention allows for accurate measurement of intraocular pressure while addressing some of the deficiencies of existing tonometers.

An applanation tonometer for flattening the cornea of an eye is provided, wherein the applanator comprises a fiberoptics array; a force transducer for measuring forces applied by the applanator; an image transducer for obtaining data regarding an image of the applanated eye; and a processing circuit configured to calculate an intraocular pressure of the eye from at least a measured force and corresponding measured geometrical property. In aspects of the preferred embodiment, the geometrical property of the image may comprise an area, diameter, or major and minor axes of the cornea of the applanated eye. The tonometer may further comprise a light source for illuminating the eye, and a lens system adapted to focus an image of the applanated portion of the eye from the applanator to the image sensor. The tonometer calculates an intraocular pressure using a plurality of corresponding forces and geometrical properties of the applanation image as more particularly described in the above Ser. No. 10/137,234. As an important aspect of a preferred embodiment, the tonometer is provided with a new form of disposable tip that covers the applanator, thereby providing an improved replaceable, sterile interface between the tonometer's applanator and a patient's eye.

Thus more, particularly, the present invention is directed to a new form of disposable tip cover for use with an applanation tonometer and which comprises a light transmitting applanating region adapted to fit over the tip of a fiber optic applanator of the tonometer. The applanating region has a surface adapted to optically couple with an applanator and another surface adapted to contact the eye, and an attachment mechanism is provided for detachably attaching the tip cover to the tip of the fiber optics array. The disposable tip cover preferably comprises a thin optically diffusing translucent film, and the film preferably is sandwiched between an inner ring which is adapted to be disposed on the fiber optic array cylinder of the tonometer, and an outer ring for securing the film onto the inner ring for facilitating and providing intimate contact of a surface of the film with the outer surface of the cylinder.

### BRIEF DESCRIPTION OF THE DRAWINGS

FIG. 1 is a plan view of an applanation tonometer illustrating a disposable tip on the distal end thereof;

FIG. 2 is a view illustrating the manner in which the disposable tip is assembled;

FIG. 3 is a cross-sectional view of the assembled disposable tip;

FIG. 4 is a top view of the tip of FIG. 3;

FIG. 5 is a cross-sectional view of the inner ring of the tip;

FIG. 6 is a cross-sectional view of the outer ring of the tip′

FIG. 7 is a top view of the inner ring; and

FIGS. 8-11 illustrate a tray within which disposable tips may be disposed ready to be dispensed.

### DETAILED DESCRIPTION

The present application is directed to a new form of disposable tip, tip cover, and method of assembly for a handheld applanation tonometer. The above-identified application discloses the details of an applanation tonometer with which the present tip can be used, and also shows and describes a similar disposable tip which is retained on an elongated fiber optic array cylinder thereof which extends outward from the tonometer.

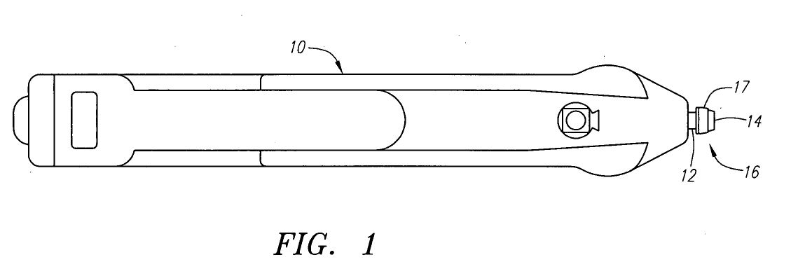

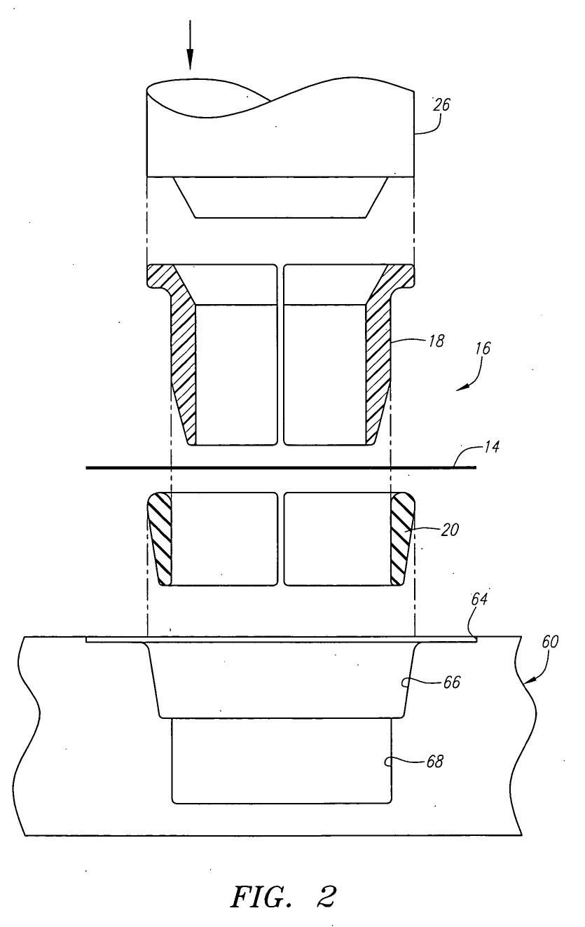

FIG. 1 illustrates an applanation tonometer 10 having a fiber optic array cylinder 12 functioning as the applanator and extending outwardly therefrom like that disclosed in the aforesaid pending application. FIG. 1 also discloses a disposable tip 16 in the form of a film 14 to be described below and which film is secured onto the cylinder 12 by a flexible ring assembly 17 comprising a pair of rings 18 and 20 in FIG. 2.

The ring assembly 17 comprises an inner ring 18 and outer ring 20 as illustrated in FIG. 2 which FIG. 1 illustrates the assembly of the film 14 onto the ring assembly 17 and which will be described in more detail subsequently.

The fiber optic array cylinder 12 provides a number of important optical and mechanical characteristics for the tonometer system. It is rigid and transfers the force of the applanation to a force sensor (not shown) within the tonometer 10 directly and without corruption. It provides collimation and therefore produces even and uninterrupted conduction of illumination to the interior surface of the tip cover 14. The optical properties of fiber optics naturally exclude ambient light which allows maintenance of even and optimized illumination for high image contrast while miminizing the effects of ambient illumination which may be present. It provides a sealed and long-protruding image conduit from the body of the tonometer 10 to allow the contact area thereof to be located some distance away from delicate interior components of the tonometer, and transfers the eye contact image through a solid interface into the interior of the instrument without distortion or loss of contrast. It provides a mechanically stable mounting surface for the disposable tip cover 14.

An active light source (not shown) within the tonometer does not illuminate the eye directly or require any fluorescent dyes, but rather illuminates the tip cover 14 distal surface. The tip cover 14 is translucent, and the tip cover provides certain unique optical characteristics. It suppresses the iris image and other image artifacts caused by reflection from structures located behind the cornea as a result of the diffusing properties of the film of the tip cover 14. The material preferably is an EMA film which is thin, preferably less than one mil, which is necessary to prevent distortion of the force signal. It is a neutral color, which helps to produce maximum image contrast. It is translucent which is necessary to allow light to be coupled out during contact with the cornea of the eye. It is diffusing to prevent spurious imaging of structures not in contact with the tonometer tip. It is impermeable to moisture which helps to maintain the tip 14 and fiber optics array 12 interface optical properties. It is non-wetting to prevent the collection of moisture and producing spurious images. It resists mechanical deformations of stretching, creasing or folding, to thereby prevent optical distortion of the contact image. It resists shearing or tearing to facilitate normal handling. It has low adhesion to allow convenient handling and avoid adhesion to the fiber optics array 12. It is biocompatible and non-allergenic and non-irritating which facilitates minimizing adverse reaction with a patient's cornea. It is low in cost to allow economic disposability, and disposable to prevent cross-contamination. It maintains its properties after sterilization, and allows radiation (gamma) sterilization to prevent infection. The present system with the tip cover 14 does not require the use of any imaging dyes as with other systems.

Preferably the tip cover film 14 is Ethyl Acrylate Copolymer film, preferably about 0.9 mil. in thickness. An example is Chevron Ethylene Methyl Acrylate Copolymer grade SP2255. The material is translucent and optically diffusing, thereby reducing depth of field of the imaging system while still passing a contact eye image. This material is very tough for its thickness and provides a barrier between the patient contact area and the tonometer tip 12. The tip cover 14 is meant to be low cost and disposable. It provides protection of the patient during patient to patient procedures.

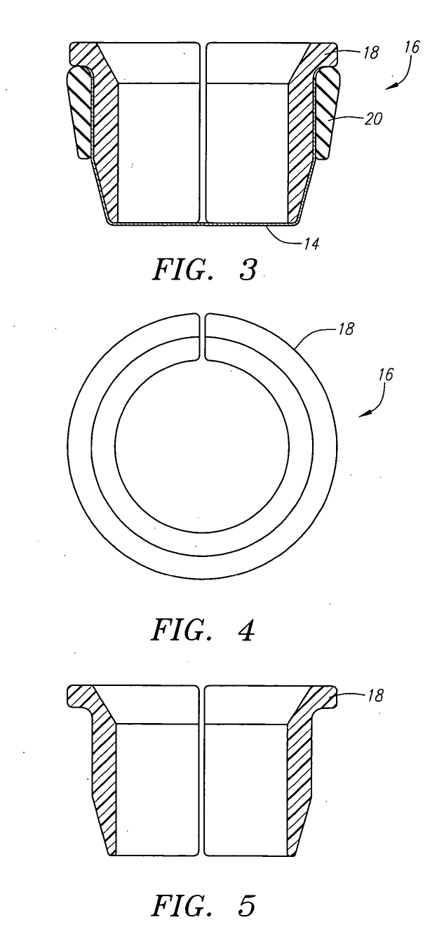

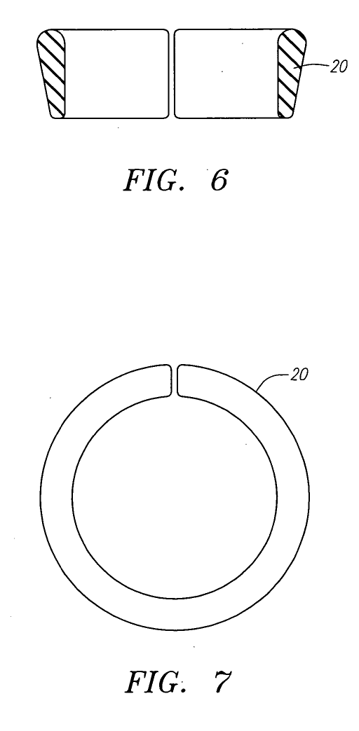

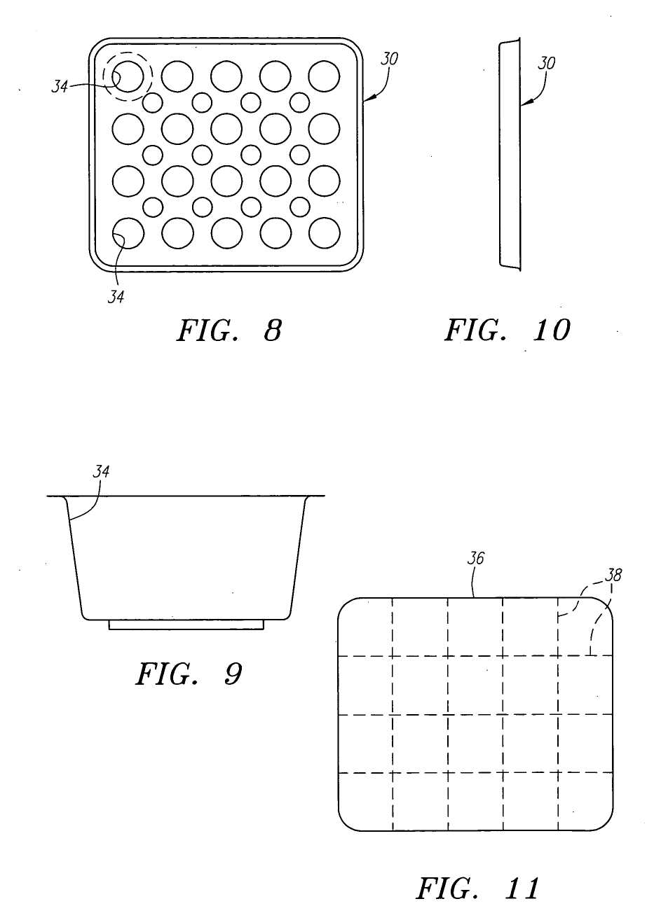

Turning back to the drawings, FIGS. 3 through 7 illustrate the inner ring 18 and the outer ring 20 in detail. The outer ring serves to secure the film 14 onto the inner ring 18 of the tip 16. FIGS. 8 through 11 illustrate an exemplary vacuum-formed tray for holding and storing the tip 16 after assembly which is described below.

Turning again to FIG. 2, the same illustrates an assembly tool 60 for facilitating assembly of the film tip 14 and ring assembly 17 to form the tip 16. This tool 60 includes an upper cavity 64 for receiving a circular piece of the film 14 which along with ring 17 will result in the ultimate disposable tip 16. The tool 60 includes a next lower cavity 66 into which the outer ring 20 is disposed or nested, and a lower or bottom cavity. In assembly, the outer ring is placed in the cavity 66, followed by the circular piece of EMA film 14 into the upper cavity 64 on top of the outer ring 20.

The inner ring 18 is attached to a mandrel 26. The mandrel is concentrically aligned with the cavities 64, 66 and 68 such that when lowered into the cavities, the inner ring 18 pushes the EMA film 14 over the outer diameter of the inner ring 18 as the inner ring is gradually pushed into the outer ring 20. The desired result is for the EMA film to be tightly secured between the two rings 18 and 20.

In this new 3-piece device the tip cover 17 must first be assembled with the film 14 as described above. After the tip cover is assembled to form the tip cover 16, it is placed into a cavity in a plastic clear copolyester compartmentalized tray 30 of FIG. 8. To install the tip 16 on the fiber-optic tip of the tonometer, the operator aligns the fiber tip 12 with the inside diameter of the inner ring 18 and presses the fiber tip into the inner ring causing the tip cover to expand slightly (due to the spring action inherent in the split inner and outer rings), gripping the fiber tip firmly. The device is then removed from the plastic tray and is ready for use.

Thus, the film 14 is sandwiched between the outer ring 20 and inner ring 18 followed by application of the assembly onto the distal tip of the film cylinder 12.

Returning again to FIGS. 8 through 11, FIG. 8 illustrates the tray 30 which is used for storage of the tip cover 17. FIG. 9 illustrates an enlarged view of one of the twenty cavities 34 in the tray 30 each for holding a tip 16, and FIG. 11 illustrates a Tyvek lid for the tray 30 and which is scored at 38 to allow access to individual circular tips.

While embodiments of the present invention have been shown and described, various modifications may be made without departing from the scope of the present invention, and all such modifications and equivalents are intended to be covered.

1. A disposable tip cover for a tonometer comprising a thin optically diffusing translucent film, the film being sandwiched between an inner ring which is adapted to be disposed on a fiber optic array cylinder of a tonometer, and an outer ring for securing the film onto the inner ring for facilitating intimate contact of a surface of the film with a surface of the cylinder.

2. A tip as in claim 1 wherein a tip cover suppresses an image of the iris of the eye and other image artifacts caused by reflection from structures located behind the cornea of the eye.

3. A tip as in claim 1 wherein the tip cover is an EMA film, preferably one mil or less thick for preventing distortion of a force signal during applanation with a tonometer.

4. A tip as in claim 1 wherein the tip cover has a neutral color to help produce maximum image contrast.

5. A tip as in claim 1 wherein the tip cover is translucent to allow light to be coupled from the area of corneal contact during contact with the cornea.

6. A tip as in claim 1 wherein the tip cover is diffusing to prevent spurious imaging of structures not in contact with the tonometer tip.

7. A tip as in claim 1 wherein the tip cover film is ethylacrylate copolymer film, preferably about 0.9 mil in thickness.

8. A disposable tip cover for a tonometer comprising a tip cover formed of a material for reducing depth of field of the imaging system of the tonometer while passing a contact eye image to a sensor of the tonometer.

9. A tip cover as in claim 8 wherein the tip cover is translucent for preventing spurious imaging of structures of the eye not in contact with the tonometer tip.

10. A tip cover as in claim 8 further including an inner ring and an outer ring for sandwiching the tip cover therebetween.

11. A tip cover as in claim 10 wherein at least one of the rings is flexible.

12. An applanation tonometer comprising
a housing adapted to be hand held,
a fiber optic array extending from the housing and for transferring force of applanation to a force sensor within the tonometer housing and for providing collimation and even and uninterrupted conduction of illumination to an exterior tip of the cylinder, and
a disposable tip cover attachable to the exterior tip of the cylinder, the tip cover comprising a translucent and light diffusing film.

13. A tonometer as in claim 12 wherein the tip cover is an EMA film, preferably one mil or less thick for preventing distortion of a force signal during applanation with a tonometer.

14. A tonometer as in claim 4 wherein the tip cover is an EMA film, preferably one mil or less thick for preventing distortion of a force signal during applanation with a tonometer.

15. A tonometer as in claim 7 wherein the tip cover is an EMA film, preferably one mil or less thick for preventing distortion of a force signal during applanation with a tonometer.
